(19) United States
(12) Patent Application Publication
(10) Pub. No.: US 2025/0128025 A1
(43) Pub. Date: Apr. 24, 2025

(54) TARGET MOVEMENT MODELLING USING ELECTROMAGNETIC NAVIGATION SENSORS

(71) Applicant: Covidien LP, Mansfield, MA (US)

(72) Inventors: Guy Alexandroni, Yehud-Monosson (IL); Irina Shevlev, Ashdod (IL)

(73) Assignee: Covidien LP, Mansfield, MA (US)

(21) Appl. No.: 18/895,020

(22) Filed: Sep. 24, 2024

Related U.S. Application Data
(60) Provisional application No. 63/545,207, filed on Oct. 23, 2023.

(51) Int. Cl. A61M 25/01; A61B 17/00; A61B 34/10; A61B 34/20

(52) U.S. Cl. CPC A61M 25/0127; A61B 2017/00699; A61B 2034/107; A61B 2034/2051; A61M 2025/0166

(57) ABSTRACT

Systems and methods for target movement modelling use sequences of position data from a first electromagnetic (EM) sensor disposed at a distal portion of a catheter disposed in a lung and from at least one second EM sensor disposed at the patient's chest in order to update target tissue position. The methods involve generating a breathing model of the lungs as a function of breathing phases based on the position data from the first and second EM sensors. The methods also involve receiving current position data from the at least one second EM sensor and estimating a current breathing phase based on the breathing model and the current position data. The methods also involve predicting displacement of the target based on the breathing model and the current breathing phase, and updating body coordinates near the target based on the displacement of the target.

### FIELD

The technology of this disclosure is generally related to modeling target tissue movement based on position information from one or more electromagnetic (EM) sensors in order to correct local patient body coordinates.

### BACKGROUND

When performing a medical procedure, clinicians often rely on patient data including X-ray data, computed tomography (CT) scan data, magnetic resonance imaging (MRI) data, or other imaging data that allows the clinician to view the internal anatomy of a patient. The imaging data is also utilized to identify targets of interest and to develop strategies for accessing the targets of interest for surgical treatment. Further, the imaging data has been used to create a three-dimensional (3D) model of the patient's body to guide navigation of the medical device to a target of interest within a patient's body.

Since it is important to treat a target at an exact location from a planned direction, even a small discrepancy between the actual location and an estimated location of the medical device may cause undesired consequences in the medical procedure. Thus, precision in estimating the actual location of the medical device with sufficient level of accuracy is highly desirable during medical procedures.

Further, when the medical device approaches the target following the 3D model, a patient's breathing and heartbeat cause the medical device to appear to move in the 3D model even though the medical device is stably positioned with respect to internal organs surrounding the target within the patient's body. Thus, stabilizing the respiratory movements is beneficial in properly displaying the location of the medical device during medical procedures.

### SUMMARY

The techniques of this disclosure generally relate to modeling target tissue movement based on position information from one or more electromagnetic (EM) sensors in order to correct local patient body coordinates.

In one aspect, the disclosure provides a method of updating a position of a target based on the compatible movement of the target. The method includes determining movement of a catheter disposed in a lung during at least one breathing cycle of a patient and generating a model of movement of a target based on the movement of the catheter. The method also includes determining movement of at least one PST during at least one breathing cycle of the patient and generating a model of movement of the chest of the patient based on the movement of the at least one PST. The method also includes receiving a live PST signal from the at least one PST and estimating breathing phase based on the model of the movement of the chest and the live PST signal. The method also includes estimating a movement of the target compatible with the model of the movement of the target based on the breathing phase, yielding a compatible movement of the target, and updating a position of a target based on the compatible movement of the target.

In aspects, implementations of the method of updating a position of a target based on the compatible movement of the target may include one or more of the following features. The position of the target may be updated in coordinates of the body of the patient. The method may include updating a position of the catheter based on the updated position of the target. The method may include updating the position of the target according to the breathing phase. The method may include filtering the live PST signal to remove frequencies outside of a normal breathing frequency range.

Determining movement of the catheter may include receiving position data from at least one EM sensor disposed at an end portion of the catheter during at least one breathing cycle of the patient and filtering the position data to remove position values outside of a predetermined position value range. The method may include determining that an amplitude of position data of the catheter during a breathing cycle is greater than a threshold, and not updating the position of the target in response to determining that the amplitude of position data of the catheter during the breathing cycle is greater than a threshold.

In another aspect, the disclosure provides a method of updating body coordinates near the target based on the displacement of the target. The method includes generating a breathing model of the lungs as a function of breathing phases based on position data from a first electromagnetic (EM) sensor disposed at a distal portion of a catheter disposed in a lung of a patient and from at least one second EM sensor disposed on the chest of the patient. The method also includes receiving current position data from the at least one second EM sensor and estimating a current breathing phase based on the breathing model and the current position data. The method also includes predicting displacement of the target relative to an average position of the target based on the breathing model and the current breathing phase, and updating body coordinates near the target based on the displacement of the target.

In aspects, implementations of the method of updating body coordinates near the target based on the displacement of the target may include one or more of the following features. The at least one second EM sensor may be at least one PST. Generating the breathing model of the lungs may include generating a model of chest movement as a function of breathing phase based on the position data from the first EM sensor, generating a model of target movement as a function of breathing phase based on the position data from the at least one second EM sensor, and combining the model of the chest movement and the model of the target movement to obtain the breathing model of the lungs. The receiving the current position data, the estimating the current breathing phase, the predicting the displacement of the target, and the updating the body coordinates may be performed during a navigation procedure, a biopsy procedure, or an ablation procedure.

The method may include displaying a message to the user to navigate the catheter near the target. The method may include displaying a message to the user to not move the catheter. The method may include simultaneously recording the position data from the first EM sensor and from the at least one second EM sensor during at least one breathing cycle of the patient.

In still another aspect, the disclosure provides a system for updating body coordinates near the target tissue based on the displacement of the target tissue. The system includes a catheter configured to be placed near target tissue in a lung of a patient and a first electromagnetic (EM) sensor disposed at a distal portion of the catheter, and at least one second EM sensor disposed on the chest of the patient. The system also includes a processor and a memory having stored thereon instructions, which, when executed by the processor, cause the system to receive first position data from the first EM sensor, receive second position data from the at least one second EM sensor, and generate a breathing model of the lungs as a function of breathing phases based on the first position data and the second position data.

The instructions, when executed by the processor, also cause the system to receive current position data from the at least one second EM sensor and estimate a current breathing phase based on the breathing model and the current position data. The instructions, when executed by the processor, also cause the system to predict displacement of the target tissue based on the breathing model and the current breathing phase, and update body coordinates near the target tissue based on the displacement of the target tissue.

In aspects, implementations of the system may include one or more of the following features. The instructions, when executed by the processor, may cause the system to generate a model of lung tissue movement as a function of breathing phase based on the position data from the first EM sensor, generate a model of chest movement as a function of breathing phase based on the position data from the at least one second EM sensor, and generate the breathing model of the lungs based on the model of the target tissue movement and the model of the chest movement.

The instructions, when executed by the processor, may cause the system to receive the current position data, estimate the current breathing phase, predict the displacement of the target tissue, and update the body coordinates during a navigation procedure, a biopsy procedure, or an ablation procedure. The system may include a display, and the instructions, when executed by the processor, may cause the display to display a message to the user to navigate the catheter near the target tissue.

The instructions, when executed by the processor, may cause the system to simultaneously record the first position data and the second position data during at least one breathing cycle of the patient. The instructions, when executed by the processor, may cause the system to register coordinates of the at least one second EM sensor to the coordinates of the body of the patient, yielding a sensor to body registration; determine that movement of lung tissue is less than a threshold; and correct the sensor to body registration in response to determining that movement of lung tissue is less than the threshold.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram that illustrates a system for acquiring and processing 3D CBCT scans of a patient and a location board;

FIG. 2 is a circuit block diagram that illustrates the workstation of the system of FIG. 1;

FIG. 3 is a flowchart that illustrates an example of a method for correcting a position of a catheter and/or target tissue near the catheter;

FIG. 4 is a flowchart that illustrates an example of a method for generating a breathing model; and

FIG. 5 is a flowchart that illustrates another example of a method for correcting a position of a catheter and/or target tissue near the catheter.

### DETAILED DESCRIPTION

During an Electromagnetic Navigation Bronchoscopy (ENB) procedure, the lung tissue continuously moves as a result of the patient's breathing and heart beats. When the lungs inhale, the diaphragm contracts and pulls downward towards the abdominal cavity. At the same time, the intercostal muscles (the muscles between the ribs) contract and pull the rib cage upward. This increases the size of the thoracic cavity and decreases the pressure inside the thoracic cavity. As a result, air rushes in and fills the lungs. When the lungs exhale, the diaphragm relaxes, and the volume of the thoracic cavity decreases, while the pressure within the thoracic cavity increases. As a result, the lungs contract and air is forced out of the lungs.

As a result, a point of interest or a target in the lungs has a periodic movement. On average the movement amplitude along the main movement direction is about 2.5 mm but can reach up to 1.5 cm and even more. The movement amplitude and direction is a function of the target position in the lung (which lobe, proximity to rigid structures, etc.)

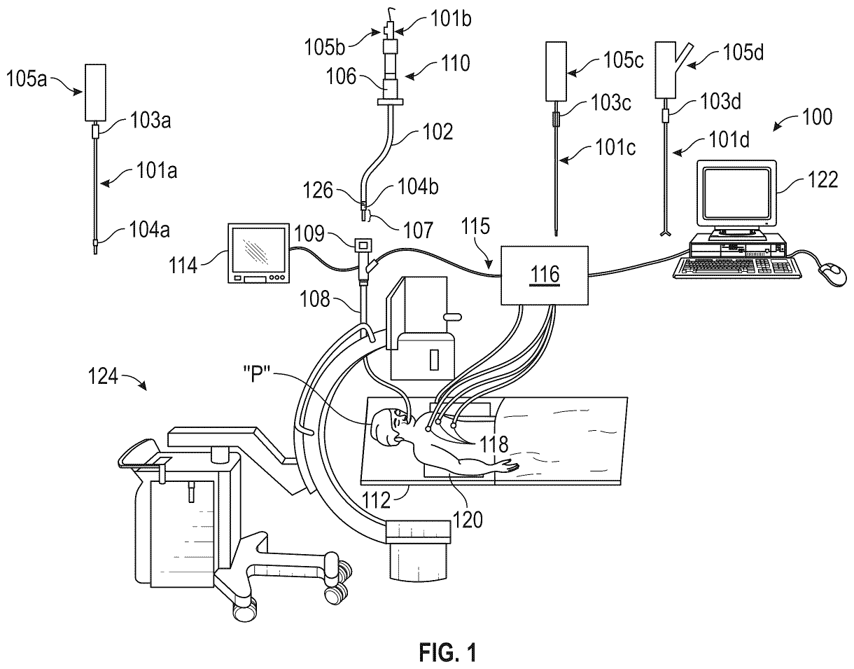

Medtronic's Illumisite navigation system continuously tracks a location of a navigation sensor, e.g., sensor 104a illustrated in FIG. 1, in the airways of the lungs. The navigation sensor location is first solved in antenna coordinates. Next, as shown in FIG. 1, the navigation sensor location is transformed to estimated body coordinates based on continuous tracking of the patient sensor triplet (PST) 118. The PST 118 senses general chest movement and can sense the chest breathing movement; however, the diaphragm movement is oblivious to the PST 118. Hence, most of the breathing movement is unaccounted for. Also, lung tissue movement due to heart beats may not be sensed. As a result, the navigation catheter location inside the airway tree is inaccurate. For example, the target may appear static as the navigation catheter continuously moves due to the breathing and/or heart beating activity, while in reality both the target and the navigation catheter move.

As another example, in target overlay, the estimated target location is projected onto the live fluoroscopy image; however, the uncompensated body movement causes an inaccurate target overlay. The overlay may remain relatively static as the anatomy under the overlay and the target moves continuously.

In accordance with aspects of the disclosure, the navigation catheter is used to record and model movement of the target along a breathing cycle. The PST 118 is used to model the chest movement along a breathing cycle.

Breathing compensation may be accomplished by using a live PST signal to estimate a breathing phase and to estimate the compatible target movement at the estimated breathing phase. Breathing may be characterized as a continuous and periodic movement measured by the PST 118. The inhale phase and the exhale phase are the two extreme phases that may be sampled from the live PST signal. Alternatively, or additionally, a system or method may involve sampling the live PST signal with more granularity. For example, if a full breathing cycle is 6 seconds long, the phase value can be any integer between 0 and 6. In general, the phase value can be any integer between 0 and a non-negative, non-zero integer. The non-negative, non-zero integer may be increased to a desired value to achieve a desired resolution or accuracy of the breathing phase. Next, local correction may be created in body coordinates and may be updated constantly according to the breathing phase. Then, the catheter position and/or the target position may be updated according to the local correction.

FIG. 1 is a perspective view of an example of a system 100 for facilitating navigation of a medical tool, e.g., a catheter, to a target via airways of the lungs. The system 100 may be further configured to construct radiographic-based volumetric data of the target area from intraprocedural 2D radiographic images, e.g., intraprocedural fluoroscopic and/or CBCT images, to confirm navigation of an sEWC to a desired location near the target area, where a tool may be placed through and extending out of the sEWC. In aspects, the imaging system 124 of the system 100 may include one or more of a C-arm fluoroscope, a 3D cone-beam computed tomography (CBCT) imaging system, and a 3D fluoroscopic imaging system.

The system 100 may be further configured to facilitate approach of a medical tool or tool to the target area and to determine the location of the medical tool with respect to the target by using electromagnetic navigation (EMN) of the sEWC. One such EMN system is the ILLUMISITE system currently sold by Medtronic PLC, though other systems for intraluminal navigation are considered within the scope of this disclosure.

One aspect of the system 100 is a software component for reviewing computed tomography (CT) image scan data that has been acquired separately from the system 100. The review of the CT image data allows a user to identify one or more targets, plan a pathway to an identified target (planning phase), navigate the sEWC 102 to the target (navigation phase) using a user interface running on a computer system 122, and confirming placement of a distal end portion of the sEWC 102 near the target using one or more electromagnetic (EM) sensors 104b, 126 disposed in or on the sEWC 102 at a predetermined position at or near the distal end portion of the sEWC 102. While this disclosure refers to the sEWC 102 with one or more EM sensors 104b, 126, this disclosure contemplates use of any suitable endoluminal device, which includes one or more localization sensors (e.g., either or both of EM sensors 104b, 126) at the distal tip or end portion of the endoluminal device, and which can be navigated to a target area of a lung via airways of the lung. For example, the sEWC 102 may be replaced by an EWC or other suitable catheter incorporating a localization sensor and capable of being navigated to a target area of a lung via airways of the lung. The localization sensor may be any suitable sensor that provides x,y,z coordinates.

The target may be tissue of interest identified by review of the CT image data during the planning phase. Following navigation of the sEWC 102 near the target, a medical tool, such as a biopsy tool, an access tool, or a therapy tool, e.g., a flexible microwave ablation catheter, is inserted into and fixed in place with respect to the sEWC 102 such that a distal end portion of the medical tool extends a desired distance 107 beyond the distal end of the sEWC 102 and the sEWC 102 is further navigated using EM navigation to obtain a tissue sample, enable access to a target site, or apply therapy to the target using the medical tool.

As shown in FIG. 1, the sEWC 102 is part of a catheter guide assembly 110. In practice, the sEWC 102 is inserted into a bronchoscope 108 for access to a luminal network of the patient P. Specifically, the sEWC 102 of catheter guide assembly 110 may be inserted into a working channel of bronchoscope 108 for navigation through a patient's luminal network. A bronchoscope adapter 109 is coupled to the proximal end portion of the bronchoscope. The bronchoscope adapter 109 may be the EDGE™ Bronchoscope Adapter, which is currently marketed and sold by Medtronic PLC. The bronchoscope adapter 109 is configured either to allow motion of the sEWC 102 through the working channel of the bronchoscope 108 (which may be referred to as an unlocked state of the bronchoscope adapter 109) or prevent motion of the sEWC 102 through the working channel of the bronchoscope (which may be referred to as an unlocked state of the bronchoscope adapter 109).

Aspects of the disclosure may be applied to a variety of procedures including biopsy, ablation, or marker placement procedures. For example, the procedures may involve one or more of a locatable guide 101a, a microwave ablation tool 101b, a biopsy needle 101c, or a forceps 101d. The locatable guide (LG) 101a, which may be a catheter, and which may include a sensor 104a similar to the sensor 104b, is inserted into the sEWC 102 and locked into position such that the sensor 104a extends a predetermined distance beyond the distal end portion of the sEWC 102. The tools 101a-101d of the same lengths which include a fixing member 103a-d such that when the fixing member 103a-103d of the tools 101a-101d engages, e.g., snaps in, with the proximal end portion of the handle 106 of the catheter guide assembly 110, the LG 101a extends a predetermined distance 107 beyond a distal tip or end portion of the sEWC 102. The predetermined distance 107 may be based on the length of the sEWC 102 and a length between the end portion of the handle 105a-d or the fixing member 103a-103d and the distal end portion of the LG 101a or the other medical tools 101b-101d. In aspects, the handles 105a-105d may include control objects, e.g., a button or a lever, for controlling operation of the medical tools 101a-101d.

In some aspects, the position of the fixing member 105a-105d along the length of the medical tools 101a-101d may be adjustable so that the user can adjust the distance by which the distal end portion of the LG 101a or the medical tools 101b-101d extend beyond the distal end portion of the sEWC 102. The position and orientation of the LG sensor 104a relative to a reference coordinate system within an electromagnetic field can be derived using an application executed by the computer system 122. In some aspects, the sEWC 102 may act as the LG 101a, in which case the LG 101a may not be used. In other aspects, the sEWC 102 and the LG 101a may be used together. For example, data from the sensors 104a and 104b may be fused together. Catheter guide assemblies 110 are currently marketed and sold by Medtronic PLC under the brand names SUPERDIMENSION® Procedure Kits, or EDGE™ Procedure Kits, and are contemplated as useable with the disclosure.

The system 100 generally includes an operating table 112 configured to support a patient P; a bronchoscope 108 configured for insertion through patient P′s mouth into patient P′s airways; monitoring equipment 114 coupled to bronchoscope 108 (e.g., a video display for displaying the video images received from the video imaging system of bronchoscope 108); and a tracking system 115 including a tracking module 116, patient sensor triplet (PST) 118, and a location or transmitter board 120. The location board 120 includes one or more EM transmitters for generating an EM field.

The location board 120 may also include fiducials, which may be embedded or otherwise incorporated into the location board 120, and which are designed and/or arranged to appear in radiographic images for the purpose of creating a 3D reconstruction from the radiographic images. Since the fiducials may be radiographically dense, the fiducials create artifacts on the radiographic images, e.g., intraoperative 3D CBCT images. The system 100 further includes a computer system 122 on which software and/or hardware are used to facilitate identification of a target, planning a pathway to the target, navigating a medical tool to the target, and/or confirmation and/or determination of placement of the sEWC 102, or a suitable tool therethrough, relative to the target.

As noted above, an optional imaging system 124 capable of acquiring 3D CBCT images or fluoroscopic images of the patient P is also included in the system 100. The images, sequence of images, or video captured by the imaging system 124 may be stored within the imaging system 124 or transmitted to the computer system 122 for storage, processing, and display. Additionally, the imaging system 124 may move relative to the patient P so that images may be acquired from different angles or perspectives relative to patient P to create a series of 3D CBCT images.

The pose of the imaging system 124 relative to patient P and while capturing the images may be estimated via markers incorporated with the transmitter mat 120, in the operating table 112, or a pad (not shown) placed between the patient and the operating table 112. The markers are positioned under patient P, between patient P and operating table 112 and between patient P and a radiation source or a sensing unit of the imaging system 124. The markers may have a symmetrical spacing or may have an asymmetrical spacing, a repeating pattern, or no pattern at all. The imaging system 124 may include a single imaging system or more than one imaging system. When a CBCT system is employed, the captured images can be employed to confirm the location of the sEWC 102 and/or one of the medical tools 101a-101d within the patient, update CT-based 3D modeling, or replaced pre-procedural 3D modeling with intraprocedural modeling of the patient's airways and the position of the sEWC 102 within the patient.

The computer system 122 may be any suitable computer system including a processor and storage medium, such that the processor is capable of executing instructions stored on the storage medium. The computer system 122 may further include a database configured to store patient data, CT data sets including CT images, 3D CBCT images and data sets, 3D fluoroscopic data sets including 3D fluoroscopic images and video, 3D reconstructions, navigation plans, and any other such data. Although not explicitly illustrated, the computer system 122 may include inputs, or may otherwise be configured to receive, CT data sets, CBCT or fluoroscopic images or video, and other suitable imaging data. Additionally, the computer system 122 includes a display configured to display graphical user interfaces. The computer system 122 may be connected to one or more networks through which one or more databases may be accessed by the computer system 122.

With respect to the navigation phase, a six degrees-of-freedom electromagnetic locating or tracking system 115, or other suitable system for determining position and orientation of a distal portion of the sEWC 102 (e.g., Fiber-Bragg flex sensors), is utilized for performing registration of pre-procedure images (e.g., a CT image data set and 3D models derived therefrom) and the pathway for navigation with the patient as they are located on operating table 112.

In an EMN-type system, the tracking system 115 may include the tracking module 116, the PST 118, and the location board 120 (including the markers). The tracking system 115 is configured for use with a locatable guide, and particularly the LG sensor. As described above, the medical tools, e.g., the locatable guide 101a with the LG sensor 104a, are configured for insertion through the sEWC 102 into patient P's airways (either with or without the bronchoscope 108) and are selectively lockable relative to one another via a locking mechanism, e.g., the bronchoscope adapter 109. The transmitter mat 120 is positioned beneath patient P. The transmitter mat 120 generates an electromagnetic field around at least a portion of the patient P within which the position of the LG sensor 104a, the sEWC sensor 104b, and the PST 118 can be determined through use of a tracking module 116. An additional electromagnetic sensor 126 may also be incorporated into the end of the sEWC 102. The additional electromagnetic sensor 126 may be a five degree-of-freedom sensor or a six degree-of-freedom sensor. One or more of the reference sensors of the PST 118 are attached to the chest of the patient P.

Registration refers to a method of correlating the coordinate systems of the pre-procedure images, and particularly a 3D model derived therefrom, with the patient P's airways as, for example, observed through the bronchoscope 108 and allow for the navigation to be undertaken with accurate knowledge of the location of the LG sensor within the patient and an accurate depiction of that position in the 3D model. Registration may be performed by moving the LG sensor through the airways of the patient P. More specifically, data pertaining to locations of the LG sensor, while the locatable guide is moving through the airways, is recorded using the transmitter mat 120, the PST 118, and the tracking system 115. A shape resulting from this location data is compared to an interior geometry of passages of the 3D model generated in the planning phase, and a location correlation between the shape and the 3D model based on the comparison is determined, e.g., utilizing the software on the computer system 122. The software aligns, or registers, an image representing a location of LG sensor with the 3D model and/or two-dimensional images generated from the three-dimension model, which are based on the recorded location data and an assumption that LG remains located in non-tissue space in patient P's airways. Alternatively, a manual registration technique may be employed by navigating the bronchoscope 108 with the LG sensor to pre-specified locations in the lungs of the patient P, and manually correlating the images from the bronchoscope 108 to the model data of the 3D model.

Though described herein with respect to EMN systems using EM sensors, the instant disclosure is not so limited and may be used in conjunction with flexible sensor, shape sensors such as Fiber-Bragg gratings, ultrasonic sensors, or any other suitable sensor that does not emit harmful radiation. Additionally, the methods described herein may be used in conjunction with robotic systems such that robotic actuators drive the sEWC 102 or bronchoscope 108 proximate the target.

At any point during the navigation process, tools such as a locatable guide 101a, a therapy tool (e.g., a microwave ablation tool 101b or a forceps 101d), a biopsy tool (e.g., a biopsy needle 101c), may be inserted into and fixed in place relative to the sEWC 102 to place one of the tools 101a-101d proximate the target using position information from the sEWC 102. The position information from the sensors 104b and/or 126 of the sEWC 102 may be used to calculate the position of the distal tip or distal end portion of any of the tools 101a-101d.

To ensure the accuracy of the position calculations, the tools 101a-101d are each designed to extend a predetermined distance from the distal end of the sEWC 102 and at least the distal portions of the tools 101a-101d that extend from the sEWC 102 are designed to be rigid or substantially rigid. The predetermined distance may be different depending on one or more of the design of the tools 101a-101d, the stiffnesses of the tools 101a-101d, or how each of the tools 101a-101d interact with different types of tissue. The tools 101a-101d may be designed or characterized to set the predetermined distance to ensure deflection is managed (e.g., minimized) so that the virtual tools and environment displayed to a clinician are an accurate representation of the actual clinical tools and environment.

Calculating the position of the distal end portion of any of the tools 101a-101d may include distally projecting the position information from the sensors 104b and/or 126 according to tool information. The tool information may include one or more of the shape of the tool, the type of tool, the stiffness of the tool, the type or characteristics of the tissue to be treated by the tool, or the dimensions of the tool.

With respect to the planning phase, the computer system 122, or a separate the computer system not shown, utilizes previously acquired CT image data for generating and viewing a 3D model or rendering of patient P's airways, enables the identification of a target (automatically, semi-automatically, or manually), and allows for determining a pathway through patient P's airways to tissue located at and around the target. More specifically, CT images acquired from CT scans are processed and assembled into a 3D CT volume, which is then utilized to generate a 3D model of patient P's airways. The 3D model may be displayed on a display associated with the computer system 122, or in any other suitable fashion. Using the computer system 122, various views of the 3D model or enhanced two-dimensional images generated from the 3D model are presented. The enhanced two-dimensional images may possess some 3D capabilities because they are generated from 3D data. The 3D model may be manipulated to facilitate identification of target on the 3D model or two-dimensional images, and selection of a suitable pathway through patient P's airways to access tissue located at the target can be made. Once selected, the pathway plan, the 3D model, and the images derived therefrom, can be saved, and exported to a navigation system for use during the navigation phase(s). The ILLUMISITE software suite currently sold by Medtronic PLC includes one such planning software.

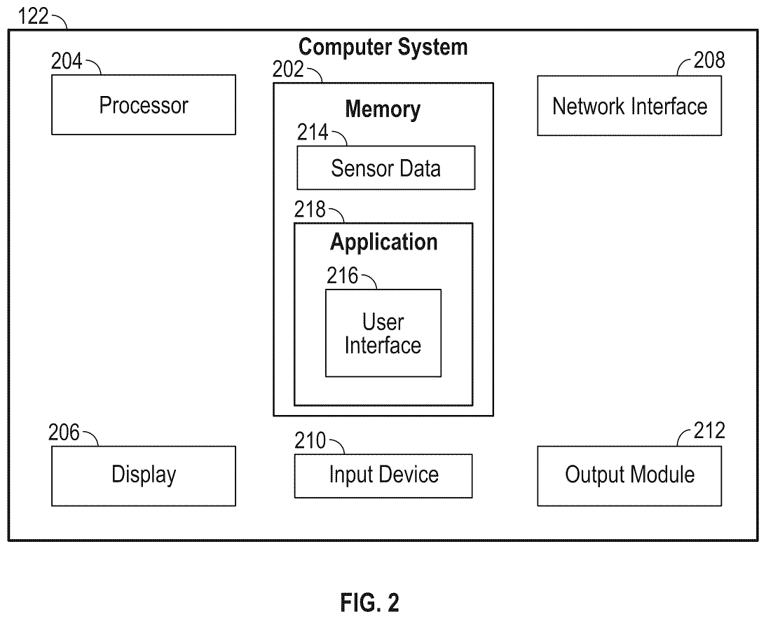

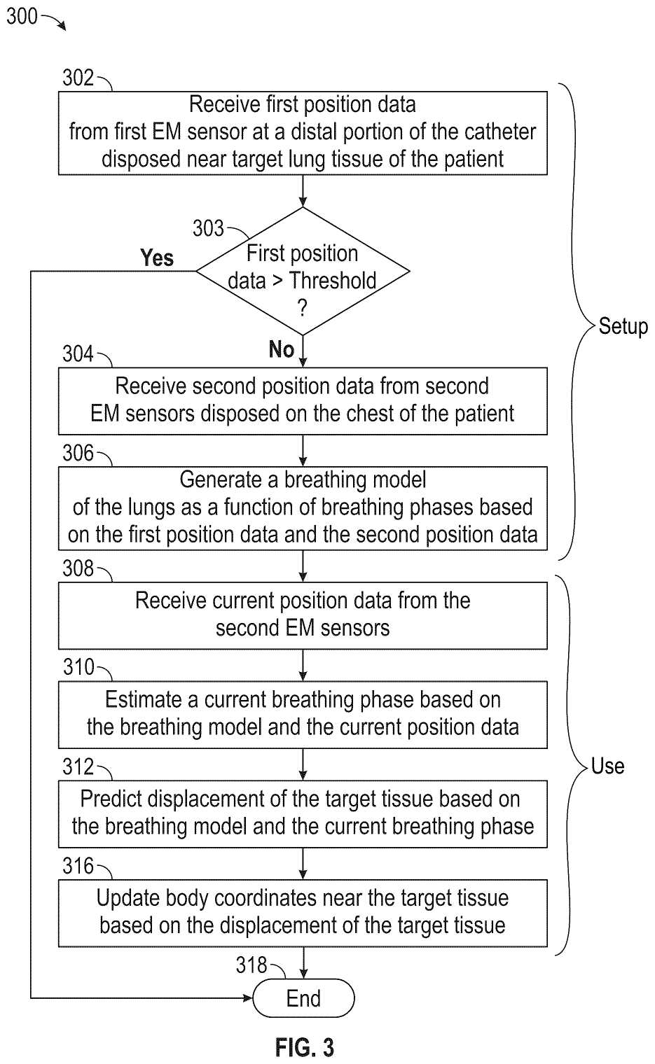

Reference is now made to FIG. 2, which is a schematic diagram of the computer system 122 of FIG. 1 configured for implementing the methods of the disclosure including the methods of FIG. 2. The computer system 122 may include a workstation. In some aspects, the computer system 122 may be coupled with the imaging system, directly or indirectly, e.g., by wireless communication. The computer system 122 may include a memory 202, a processor 204, a display 206 and an input device 210. The processor 204 may include one or more hardware processors. The computer system 122 may optionally include an output module 212 and a network interface 208. The memory 202 may store an application 218 and sensor data 214 including data from the one or more EM sensors disposed at the distal portion of the catheter and the EM sensors of the PST 118. The application 218 may include instructions executable by the processor 204 for executing the methods of the disclosure including the methods of FIGS. 3-5.

The application 218 may further include a user interface 216. The image data may include preoperative CT image data, intraoperative 3D fluoroscopic image data, preoperative or intraoperative 3D CBCT image data, and/or 3D reconstruction data. The processor 204 may be coupled with the memory 202, the display 206, the input device 210, the output module 212, the network interface 208, and the imaging system. The computer system 122 may be a stationary computer system, such as a personal computer, or a portable computer system such as a tablet computer. The computer system 122 may embed multiple computers.

The memory 202 may include any non-transitory computer-readable storage media for storing data and/or software including instructions that are executable by the processor 204 and which control the operation of the computer system 122, process data from one or more EM sensors disposed in or on the sEWC, e.g., at a distal end portion of the sEWC, to track the position of the sEWC and calculate or project the position of a distal end portion of a medical tool at a fixed position within the sEWC, and, in some aspects, may also control the operation of the imaging system. The imaging system may be used to capture a series of preoperative CT images of a portion of a patient's body, e.g., the lungs, as the portion of the patient's body moves, e.g., as the lungs move during a respiratory cycle. Optionally, the imaging system may include a 3D CBCT imaging system or a 3D fluoroscopic imaging system that captures a series of images based on which a 3D reconstruction is generated and/or to capture a live 2D view to confirm placement of the sEWC and/or the medical tool. In one aspect, the memory 202 may include one or more storage devices such as solid-state storage devices, e.g., flash memory chips. Alternatively, or in addition to the one or more solid-state storage devices, the memory 202 may include one or more mass storage devices connected to the processor 204 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 204. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by computer system 122.

The application 218 may, when executed by the processor 204, cause the display 206 to present the user interface 216. The user interface 216 may be configured to present to the user a single screen including a three-dimensional (3D) view of a 3D model of a target from the perspective of a tip of a medical tool, a live two-dimensional (2D) view showing the medical tool, and a target mark, which corresponds to the 3D model of the target, overlaid on the live 2D view. The user interface 216 may be further configured to display the target mark in different colors depending on whether the medical tool tip is aligned with the target in three dimensions.

The network interface 208 may be configured to connect to a network such as a local area network (LAN) consisting of a wired network and/or a wireless network, a wide area network (WAN), a wireless mobile network, a Bluetooth network, and/or the Internet. The network interface 208 may be used to connect between the computer system 122 and the imaging system 515. The network interface 208 may also be used to receive the sensor data 214. The input device 210 may be any device by which a user may interact with the computer system 122, such as, for example, a mouse, keyboard, foot pedal, touch screen, and/or voice interface. The output module 212 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

In aspects, the target tissue movement modelling may be performed and used to update body coordinates near the target tissue according to all or a portion of the method 300 illustrated in FIG. 3. The method 300 or other similar methods of the disclosure may be divided into a setup phase and a use phase. During the setup phase, the catheter is navigated close to the target, catheter sensor and/or PST data is acquired and processed, and a breathing model is generated based on the processed catheter sensor and/or PST data. During the use phase, live PST data is acquired and used to predict a current breathing cycle phase, target displacement is predicted based on the current breathing cycle phase, and local body coordinates in the vicinity of the target are updated based on the predicted target displacement. This effectively compensates for breathing and/or the heartbeat. Breathing causes continuous and periodic movement, which forms a breathing cycle. The breathing cycle may be broken down into inhale and exhale phases. In aspects, the PST signal may be sampled with more granularity to obtain more granular breathing cycle phases. For example, if a breathing cycle period is 6 seconds, the breathing cycle phases may include integer phase values ranging from 0 and 6.

Aspects of the disclosure may incorporate heartbeat information. Target movement caused by heartbeats is affected by the proximity of the target to the heart. Otherwise, the target movement caused by breathing may be the dominant movement and target movement caused by the heartbeat may be treated as noise or ignored. In aspects, only the PST localization signal may be used to deduce the breathing phase. Therefore, after recording the PST localization signal, the PST localization signal may be “cleaned” by filtering out frequencies not close to normal breathing frequency.

Before block 302 of FIG. 3, the clinician navigates close to the target with a catheter having an EM sensor at or near the tip of the catheter. In aspects, the method 300 may include displaying a message to the user (e.g., a clinician) to navigate close to the target with an EM navigation catheter. The method 300 may include displaying a message to the user not to move the catheter so that the catheter moves only due to lung tissue movement. The method 300 may include presenting, e.g., via a display, the estimated movement or amplitude of the breathing or breathing cycle so that the user may know how significant the breathing is in the vicinity of the target. At block 302, first position data is received from a first EM sensor at a distal portion of the catheter disposed near target lung tissue of the patient. The first position data may be recorded for at least one full breathing cycle.

In aspects, breathing correction may always be performed or only when the amplitude of catheter movement is greater than a threshold while the catheter is in the vicinity of the target during the setup phase. Accordingly, as an option, at block 303, the method 300 determines whether the first position data indicates catheter movement that is greater than a threshold. The threshold may be determined based on a maximum catheter movement determined based on medical data from one or more patients. If the method 300 determines that the catheter movement is greater than a threshold, the method 300 ends at block 318 until the start of the next position data acquisition cycle. If the method 300 determines that the catheter movement is not greater than a threshold, the method 300 proceeds to block 304 of the setup. Additionally, or alternatively, the antenna to body registration may only be performed when there is significant lung tissue movement during the setup phase. In aspects, an assessment of the lung tissue movement in order to determine whether to perform antenna to body registration may be performed between blocks 304 and 306.

At block 304, second position data is received from second EM sensors, e.g., the PST 118, disposed on the chest of the patient. The second position data may be recorded for at least one full breathing cycle. Also, the second position data may be recorded at the same time that the first position data is recorded. To ensure that the catheter is moved only by the lung tissue, the method 300 may include displaying a message warning the clinician not to move the catheter during the recording of the first and second position data.

The second position data, e.g., the PST position data, may only be used to deduce the breathing phase. Therefore, after the second position data is recorded, the second position data can be “cleaned” by filtering out frequencies from the second position data not near normal breathing frequency ranges. The normal breathing frequency ranges or respiratory rate ranges may be predetermined based on the age of the individual. For example, the normal breathing frequency ranges may be defined as follows: Newborns (0-1 month): 30-60 breaths per minute (bpm); Infants (1-12 months): 30-40 bpm; Toddlers (1-2 years): 24-40 bpm; Preschool children (3-5 years): 22-34 bpm; School-age children (6-12 years): 18-30 bpm; Teens (13-18 years): 12-20 bpm; Adults (18+years): 12-20 bpm; and Elderly: 12-22 bpm.

The filtering may be performed by any filter suitable for filtering out frequencies outside of the normal breathing frequency. The filter may be a digital filter configured as a lowpass, high pass, or bandpass filter. The digital filter may be a Finite Impulse Response (FIR) filter or an Infinite Impulse Response (IIR) filter. Specifically, the digital filter may be a Butterworth filter, a Chebyshev filter, an Elliptic (Cauer) filter, a Bessel filter, a Parks-McClellan filter, or a Windowing filter. The normal breathing frequency may be determined on a patient-by-patient basis, based on patient data related to breathing frequency, e.g., age, anatomy, and medical history.

While the target area movement may also be affected by heart beats, the methods of this disclosure deduce the breathing phase from the PST, which is designed to sense the target area movement caused by breathing. In other words, if there is target area movement due to heart beats, the methods of this disclosure may not compensate for the target area movement well. Thus, the method 300 may separate the heart-beat-related movement from the breathing-related movement by: analyzing the second position data based on frequency, separating the second position data based on frequency, and generating separate target movement models based on the separated second position data. Separating the second position data based on frequency may include identifying a first frequency range associated with breathing-related movement and a second frequency range associated with heart-beat-related movement, and determining first components of the second position data within the first frequency range and second components of the second position data within the second frequency range.

The methods of this disclosure may generate a breathing-related movement model and a heart-beat-related movement model, which may be applied separately. As described herein, the breathing-related movement model may be based on the inferred breathing phase from the PST. For the heart-beat-related movement model, the current heart-beat phase may be estimated using a heart-beat sensor or may be deduced from the catheter localization by determining a periodic component in the catheter movement in the frequency range of heart beats.

At block 306, a breathing model of the lungs is generated as a function of breathing phases based on the first position data and the second position data. The breathing model may be a dynamic three-dimensional (3D) model over a full breathing cycle. The breathing model may also be referred to as a four-dimensional (4D) model because time is the fourth dimension of the 4D model. The breathing model may be generated based on a combination of a model of the chest movement as a function of the breathing phase and a model of the target movement as a function of the current breathing phase.

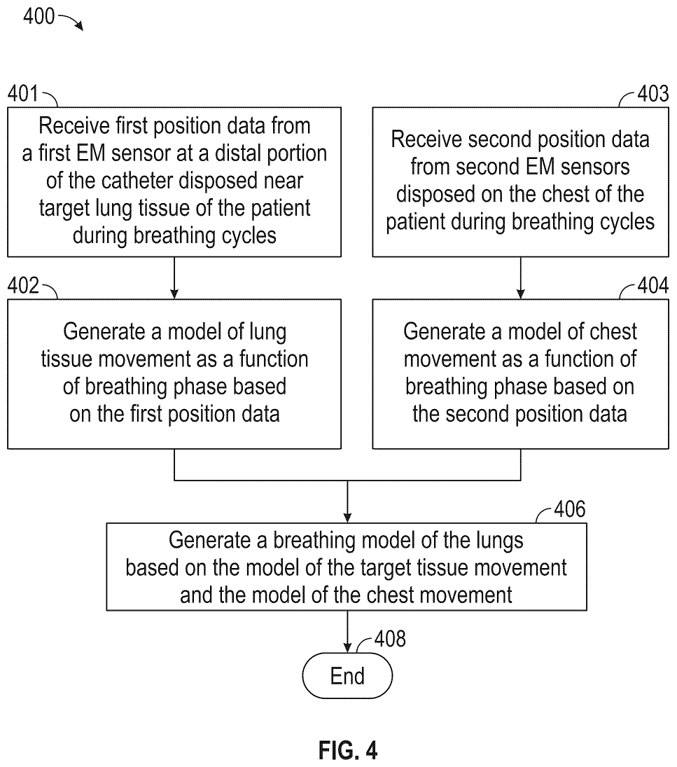

The target movement model may be generated based on the 4D localization signal of the sensor at the tip of the catheter. The target movement model is a 3D periodic function over time. The input to the target movement model is a current breathing phase and the output from the target movement model is a 3D displacement vector. The current breathing phase may be deduced by comparing the current PST position relative to a chest movement model and deduce in which phase we expect to see such a PST localization. In aspects, the breathing model may be generated according to the method 400 of FIG. 4.

FIG. 4 illustrates a method 400 of generating a breathing model. At block 401, first position data is received from a first EM sensor at a distal portion of the catheter disposed near target lung tissue of the patient during breathing cycles. At block 402, a model of lung tissue movement as a function of breathing phase based on the first position data. At block 403, second position data is received from second EM sensors disposed on the chest of the patient during breathing cycles. At block 404, a model of chest movement is generated as a function of the breathing phase based on the second position data. As illustrated in FIG. 4, blocks 401 and 402 may be executed in parallel with blocks 403 and 404. In aspects, Then, before ending at block 408, a breathing model of the lungs is generated based on the model of the target tissue movement and the model of the chest movement at block 406.

In aspects, when the model of the lung tissue movement and the model of the chest movement are used to generate a breathing model, the movement models may be weighted, for example, based on the degree of accuracy of the movement models. For example, the lung tissue movement model may be weighted more than the chest movement model. The chest movement model may be used to deduce a current breathing phase, and the breathing model may be used to deduce the target displacement at a given breathing phase.

Referring again to FIG. 3, at block 308, current position data is received from the second EM sensors. In aspects, the current position data may also be filtered in one or more of the ways described herein. At block 310, a current breathing cycle phase is estimated based on the breathing model and the current position data. The current breathing cycle phase may be any real number between zero and the breathing cycle length. At block 312, displacement of the target tissue is predicted based on the breathing model and the current breathing phase. Next, at block 316, the body coordinates near the target tissue are updated based on the displacement of the target tissue determined at block 312. Then, the method 300 ends at block 318.

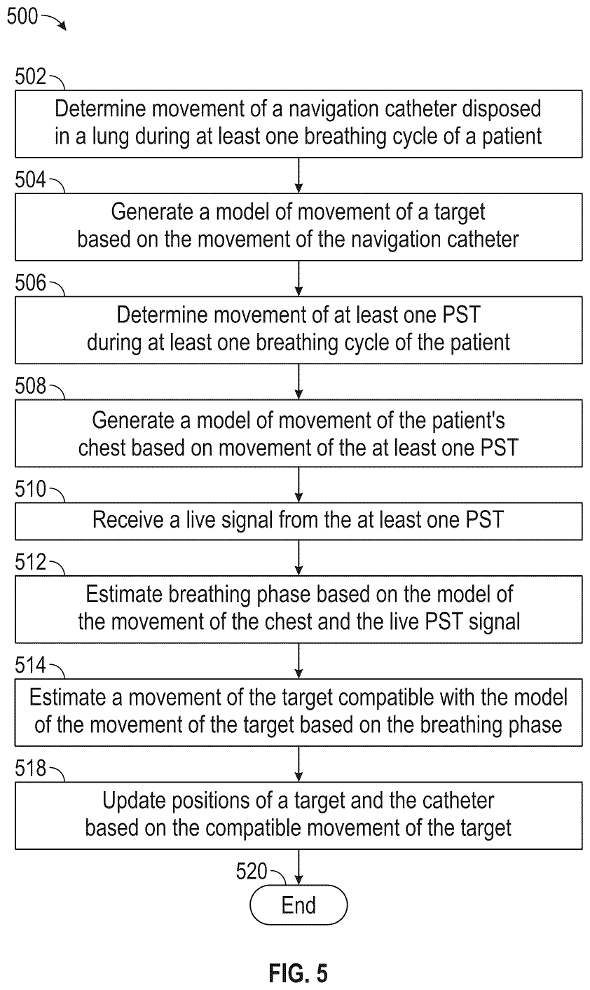

FIG. 5 illustrates another method 500 of updating positions of a target and a catheter using a breathing model based on position data from an EM sensor disposed on the catheter and a PST. At block 502, at which a setup phase starts, movement of a catheter disposed in a lung is determined during at least one breathing cycle of a patient. Movement of the catheter may be determined based on position data received from one or more EM sensors disposed at a distal portion of the catheter. At block 504, a model of movement of a target is generated based on the movement of the catheter. Block 504 may involve generating an initial 3D model of the target and surrounding tissue based on preoperative or intraoperative imaging, and generating a dynamic 3D model of the target based on the movement of the catheter within the surrounding tissue.

At block 506, movement of at least one PST is determined during at least one breathing cycle of the patient. The movement of at least one PST may be determined based on position data received from at least one PST disposed at the patient's chest. At block 508, a model of movement of the patient's chest is generated based on movement of the at least one PST. The model of the movement of the patient's chest may be a dynamic 3D model of all or a portion of the patient's thorax between the neck and the abdomen.

At block 510, at which a use phase starts, a live signal is received from the at least one PST. The live signal may be filtered prior to use. At block 512, a breathing phase is estimated based on the model of the movement of the chest and the live PST signal. At block 514, a movement of the target compatible with the model of the movement of the target is estimated based on the breathing phase. Before ending at block 520, positions of a target and the catheter are updated based on the compatible movement of the target at block 518.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The invention may be further described by reference to the following numbered paragraphs:

1. A method comprising
determining movement of a catheter disposed in a lung during at least one breathing cycle of a patient;
generating a model of movement of a target based on the movement of the catheter;
determining movement of at least one PST during at least one breathing cycle of the patient;
generating a model of movement of the chest of the patient based on the movement of the at least one PST;
receiving a live PST signal from the at least one PST;
estimating breathing phase based on the model of the movement of the chest and the live PST signal;
estimating a movement of the target compatible with the model of the movement of the target based on the breathing phase, yielding a compatible movement of the target; and
updating a position of a target based on the compatible movement of the target.

2. The method according to paragraph 1, wherein the position of the target is updated in coordinates of the body of the patient.

3. The method according to any one of the preceding paragraphs, further comprising updating a position of the catheter based on the updated position of the target.

4. The method according to any one of the preceding paragraphs, further comprising updating the position of the target according to the breathing phase.

5. The method according to any one of the preceding paragraphs, further comprising filtering the live PST signal to remove frequencies outside of a normal breathing frequency range.

6. The method according to any one of the preceding paragraphs, wherein determining movement of the catheter includes:
receiving position data from at least one EM sensor disposed at an end portion of the catheter during at least one breathing cycle of the patient; and
filtering the position data to remove position values outside of a predetermined position value range.

7. The method according to any one of the preceding paragraphs, further comprising:
determining that an amplitude of position data of the catheter during a breathing cycle is greater than a threshold; and
not updating the position of the target in response to determining that the amplitude of position data of the catheter during the breathing cycle is greater than a threshold.

8. A method comprising
generating a breathing model of the lungs as a function of breathing phases based on position data from a first electromagnetic (EM) sensor disposed at a distal portion of a catheter disposed in a lung of a patient and from at least one second EM sensor disposed on the chest of the patient;
receiving current position data from the at least one second EM sensor;
estimating a current breathing phase based on the breathing model and the current position data;
predicting displacement of the target relative to an average position of the target based on the breathing model and the current breathing phase; and
updating body coordinates near the target based on the displacement of the target.

9. The method according to paragraph 8, wherein the at least one second EM sensor is at least one PST.

10. The method according to any one of the preceding paragraphs, wherein generating the breathing model of the lungs includes:
generating a model of chest movement as a function of breathing phase based on the position data from the first EM sensor;
generating a model of target movement as a function of breathing phase based on the position data from the at least one second EM sensor; and
combining the model of the chest movement and the model of the target movement to obtain the breathing model of the lungs.

11. The method according to any one of the preceding paragraphs, wherein the receiving the current position data, the estimating the current breathing phase, the predicting the displacement of the target, and the updating the body coordinates are performed during a navigation procedure, a biopsy procedure, or an ablation procedure.

12. The method according to any one of the preceding paragraphs, further comprising displaying a message to the user to navigate the catheter near the target.

13. The method according to any one of the preceding paragraphs, further comprising displaying a message to the user to not move the catheter.

14. The method according to any one of the preceding paragraphs, further comprising simultaneously recording the position data from the first EM sensor and from the at least one second EM sensor during at least one breathing cycle of the patient.

15. A system comprising
a catheter configured to be placed near target tissue in a lung of a patient;
a first electromagnetic (EM) sensor disposed at a distal portion of the catheter;
at least one second EM sensor disposed on the chest of the patient;
a processor; and
a memory having stored thereon instructions, which, when executed by the processor, cause the system to:
   receive first position data from the first EM sensor;
   receive second position data from the at least one second EM sensor;
   generate a breathing model of the lungs as a function of breathing phases based on the first position data and the second position data;
   receive current position data from the at least one second EM sensor;
   estimate a current breathing phase based on the breathing model and the current position data;
   predict displacement of the target tissue based on the breathing model and the current breathing phase; and
   update body coordinates near the target tissue based on the displacement of the target tissue.

16. The system according to paragraph 15, wherein the instructions, when executed by the processor, further cause the system to:
generate a model of lung tissue movement as a function of breathing phase based on the position data from the first EM sensor;
generate a model of chest movement as a function of breathing phase based on the position data from the at least one second EM sensor; and
generate the breathing model of the lungs based on the model of the target tissue movement and the model of the chest movement.

17. The system according to any one of the preceding paragraphs, wherein the instructions, when executed by the processor, further cause the system to receive the current position data, estimate the current breathing phase, predict the displacement of the target tissue, and update the body coordinates during a navigation procedure, a biopsy procedure, or an ablation procedure.

18. The system according to any one of the preceding paragraphs, further comprising a display,
wherein the instructions, when executed by the processor, further cause the display to display a message to the user to navigate the catheter near the target tissue.

19. The system according to any one of the preceding paragraphs, wherein the instructions, when executed by the processor, further cause the system to simultaneously record the first position data and the second position data during at least one breathing cycle of the patient.

20. The system according to any one of the preceding paragraphs, wherein the instructions, when executed by the processor, further cause the system to:
register coordinates of the at least one second EM sensor to the coordinates of the body of the patient, yielding a sensor to body registration;
determine that movement of lung tissue is less than a threshold; and
correcting the sensor to body registration in response to determining that movement of lung tissue is less than the threshold.

What is claimed is:

1. A method comprising
determining movement of a catheter disposed in a lung during at least one breathing cycle of a patient;
generating a model of movement of a target based on the movement of the catheter;
determining movement of at least one PST during at least one breathing cycle of the patient;
generating a model of movement of the chest of the patient based on the movement of the at least one PST;
receiving a live PST signal from the at least one PST;
estimating breathing phase based on the model of the movement of the chest and the live PST signal;
estimating a movement of the target compatible with the model of the movement of the target based on the breathing phase, yielding a compatible movement of the target; and
updating a position of a target based on the compatible movement of the target.

2. The method according to claim 1, wherein the position of the target is updated in coordinates of the body of the patient.

3. The method according to claim 1, further comprising updating a position of the catheter based on the updated position of the target.

4. The method according to claim 1, further comprising updating the position of the target according to the breathing phase.

5. The method according to claim 1, further comprising filtering the live PST signal to remove frequencies outside of a normal breathing frequency range.

6. The method according to claim 1, wherein determining movement of the catheter includes:
receiving position data from at least one EM sensor disposed at an end portion of the catheter during at least one breathing cycle of the patient; and
filtering the position data to remove position values outside of a predetermined position value range.

7. The method according to claim 1, further comprising:
determining that an amplitude of position data of the catheter during a breathing cycle is greater than a threshold; and
not updating the position of the target in response to determining that the amplitude of position data of the catheter during the breathing cycle is greater than a threshold.

8. A method comprising
generating a breathing model of the lungs as a function of breathing phases based on position data from a first electromagnetic (EM) sensor disposed at a distal portion of a catheter disposed in a lung of a patient and from at least one second EM sensor disposed on the chest of the patient;
receiving current position data from the at least one second EM sensor;
estimating a current breathing phase based on the breathing model and the current position data;
predicting displacement of the target relative to an average position of the target based on the breathing model and the current breathing phase; and
updating body coordinates near the target based on the displacement of the target.

9. The method according to claim 8, wherein the at least one second EM sensor is at least one PST.

10. The method according to claim 8, wherein generating the breathing model of the lungs includes:
generating a model of chest movement as a function of breathing phase based on the position data from the first EM sensor;
generating a model of target movement as a function of breathing phase based on the position data from the at least one second EM sensor; and
combining the model of the chest movement and the model of the target movement to obtain the breathing model of the lungs.

11. The method according to claim 8, wherein the receiving the current position data, the estimating the current breathing phase, the predicting the displacement of the target, and the updating the body coordinates are performed during a navigation procedure, a biopsy procedure, or an ablation procedure.

12. The method according to claim 8, further comprising displaying a message to the user to navigate the catheter near the target.

13. The method according to claim 12, further comprising displaying a message to the user to not move the catheter.

14. The method according to claim 8, further comprising simultaneously recording the position data from the first EM sensor and from the at least one second EM sensor during at least one breathing cycle of the patient.

15. A system comprising
a catheter configured to be placed near target tissue in a lung of a patient;
a first electromagnetic (EM) sensor disposed at a distal portion of the catheter;
at least one second EM sensor disposed on the chest of the patient;
a processor; and
a memory having stored thereon instructions, which, when executed by the processor, cause the system to:
receive first position data from the first EM sensor;
receive second position data from the at least one second EM sensor;
generate a breathing model of the lungs as a function of breathing phases based on the first position data and the second position data;
receive current position data from the at least one second EM sensor;
estimate a current breathing phase based on the breathing model and the current position data;
predict displacement of the target tissue based on the breathing model and the current breathing phase; and
update body coordinates near the target tissue based on the displacement of the target tissue.

16. The system according to claim 15, wherein the instructions, when executed by the processor, further cause the system to:
generate a model of lung tissue movement as a function of breathing phase based on the position data from the first EM sensor;
generate a model of chest movement as a function of breathing phase based on the position data from the at least one second EM sensor; and
generate the breathing model of the lungs based on the model of the target tissue movement and the model of the chest movement.

17. The system according to claim 15, wherein the instructions, when executed by the processor, further cause the system to receive the current position data, estimate the current breathing phase, predict the displacement of the target tissue, and update the body coordinates during a navigation procedure, a biopsy procedure, or an ablation procedure.

18. The system according to claim 15, further comprising a display,
wherein the instructions, when executed by the processor, further cause the display to display a message to the user to navigate the catheter near the target tissue.

19. The system according to claim 15, wherein the instructions, when executed by the processor, further cause the system to simultaneously record the first position data and the second position data during at least one breathing cycle of the patient.

20. The system according to claim 15, wherein the instructions, when executed by the processor, further cause the system to:
register coordinates of the at least one second EM sensor to the coordinates of the body of the patient, yielding a sensor to body registration;
determine that movement of lung tissue is less than a threshold; and
correcting the sensor to body registration in response to determining that movement of lung tissue is less than the threshold.
